(19) United States
(12) Patent Application Publication
(10) Pub. No.: US 2025/0160709 A1
(43) Pub. Date: May 22, 2025

(54) OCULOMOTOR BASED DECEPTION DETECTION USING MOBILE DEVICE BASED SYSTEMS

(71) Applicant: Converus, Inc., Lehi, UT (US)

(72) Inventors: John C. Kircher, Lehi, UT (US); Benjamin C Stout, Lehi, UT (US); Todd K. Mickelsen, Lehi, UT (US)

(73) Assignee: Converus, Inc., Lehi, UT (US)

(21) Appl. No.: 18/517,918

(22) Filed: Nov. 22, 2023

(51) Int. Cl. A61B 5/16

(52) U.S. Cl. CPC A61B 5/164; A61B 5/163

(57) ABSTRACT

Systems and methods for test protocols for deception detection using a mobile electronic device, such as a smartphone or tablet computer that includes a camera, an audio output, such as a speaker, and a microphone. For the test, the mobile device is positioned so that the camera captures an image of at least one eye of the examinee. The examinee's face may be illuminated by a light on the mobile device and the mobile device held steady as a test is conducted. A series of “True” and “False” statements regarding relevant issues, including the issue of interest are presented to the examinee in an audio format by the mobile device. The examinee provides a spoken response when the statement concludes. The mobile device camera records data during the test. Data regarding features correlated with credibility are extracted and analyzed with a suitable model to compute the probability of deception.

### TECHNICAL FIELD

This invention relates to automated deception detection testing methods, systems, and protocols.

### BACKGROUND

U.S. Publication Patent No. 2010/0324454, the contents of which are incorporated herein by reference, discloses rapid, automated methods for using oculomotor measures to determine whether a person is being truthful or deceitful. A commercial product embodying such methods was released under the brand name EyeDetect® and has proven successful in the marketplace. Advancements in this type of testing have included those disclosed in U.S. Publication Patent No. 2021/0369162, the contents of which are incorporated herein by reference, which utilizes automated audio multi-issue comparison testing. These and other known methods have required the use of a dedicated testing station with multiple hardware components, such as a dedicated eye tracking device (infrared camera), a Windows-based computer, keyboard, mouse, and chin rest and testing may require a dedicated proctor to oversee the testing. As a result, tests have been limited in application to situations where the stations are present and required the costs of the station and proctor.

An automated deception detection testing protocol or system which utilizes mobile devices rather than a dedicated oculomotor testing and measuring station would be an improvement in the art. Such a testing method or system that removes the need for a proctor, thereby reducing examiner fatigue, corruption or bias from testing while adding standardization and objectivity in the testing process would be a further improvement in the art.

### SUMMARY

The present disclosure is directed to automated test protocols for deception detection. In one exemplary embodiment, testing equipment may include a mobile electronic device, such as a smartphone or tablet computer that includes a camera, an audio output, such as a speaker, and a microphone. A set of pre-test instructions may be provided to the examinee (who may also be referred to herein as a “test subject”) and one or more short practice sessions may be used to familiarize the examinee with the testing process.

For the test, the mobile device is positioned so that the camera captures an image of the eye or eyes of the examinee. The examinee's face may be illuminated by a light on the mobile device and the mobile device held steady as a test is conducted. In some embodiments, the mobile device may be positioned near the face of the examinee, with the rear of the device facing the examinee to allow a rear facing camera of the device to capture images of at least one of the examinee's eyes.

An automated protocol for deception detection, such as the audio multi-issue comparison test (AMCT) or the directed lie comparison test (DLC), may be used to audibly present statements to the examinee using the audio output device. Such statements may be presented as series of statements requiring a “True” or “False” response regarding the target behaviors or issues of interest. The examinee may provide a response by speaking, which is captured by the microphone of the mobile device. During the test, the camera is used to capture a series of images, allowing the measurement and recordation of eye behaviors such as pupil dilation, eye movement, and vascular activity as the True and False statements are presented and statement responses recorded. Response times may also be recorded. The system may pause the test and alert the examinee if the examinee eyes are no longer in view of the camera.

At the conclusion of the test, the recorded measurements may be analyzed using an appropriate decision model to compute the probability of credibility or deception. In some illustrative embodiments, a logistic regression equation-based decision model may be used.

In some embodiments, the examinee may self-conduct a test by positioning the mobile device for the examination, in others the examination may be conducted by a proctor who may provide instructions to the examinee and position the mobile device for the examination.

### DESCRIPTION OF THE DRAWINGS

It will be appreciated by those of ordinary skill in the art that the various drawings are for illustrative purposes only. The nature of the present disclosure, as well as other embodiments in accordance with this disclosure, may be more clearly understood by reference to the following detailed description, to the appended claims, and to the several drawings.

FIGS. 1A and 1B are images of one example of a mobile device that may be used to conduct an examination as part of a system in accordance with the present disclosure, in isolation and in position for conducting an examination with an examinee.

FIGS. 2A and 2B depict portions of an examinee's face that may be monitored by a camera on a mobile device as depicted in FIGS. 1A and 1B during testing in a testing protocol in accordance with the present disclosure.

FIG. 3 is a graph depicting data captured during an examination conducted using a system in according with the principles of the present disclosure.

FIGS. 4A and 4B are charts depicting measurement of pupil dilation captured during examinations of truthful and deceptive examinees, respectively, conducted using a system in accordance with the principles of the present disclosure.

FIGS. 5A and 5B are charts depicting measurement of pupil dilation captured during examinations of Not Credible and Credible examinees, respectively, conducted using a system in accordance with the principles of the present disclosure.

### DETAILED DESCRIPTION

The present disclosure relates to apparatus, systems, and methods for computer-implemented deception detection testing using mobile devices and audio presentation of questions and statements to the test subject. It will be appreciated by those skilled in the art that the embodiments herein described, while illustrative, are not intended to so limit this disclosure or the scope of the appended claims. Those skilled in the art will also understand that various combinations or modifications of the embodiments presented herein can be made without departing from the scope of this disclosure. All such alternate embodiments are within the scope of the present disclosure.

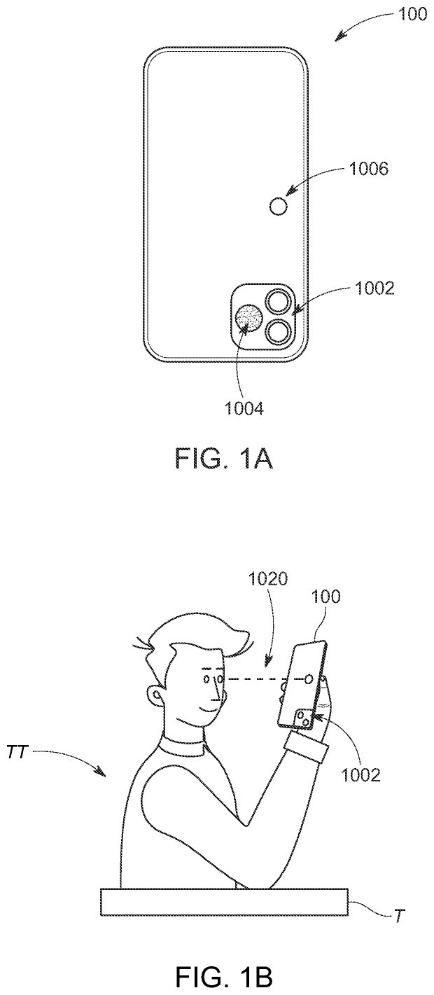

The present disclosure includes systems for conducting the testing protocols discussed herein, as well as the computer implemented methods related to such protocols. It will be appreciated that in some exemplary embodiments, such systems may include a mobile electronic device, such as a smartphone or tablet computer that includes a camera, an audio output, such as a speaker, and a microphone. FIGS. 1A and 1B depict one such illustrative mobile electronic device, a smartphone 100, which includes a camera 1002 and a light, such as flash 1004 accessible on a rear surface thereof. Mobile device 100 will also include an audio input and output, such as a microphone and speaker. A processor in the mobile device allows it to execute the instructions to operate the required components to conduct the examination, make necessary measurements, communicate with the examinee, and perform the required analysis of the collected measurements to obtain results.

As best depicted in FIG. 1B, to perform a test, the mobile device 100 is positioned so that the camera 1002 captures an image of at least one of the eyes of the examinee TT. The examinee's face may be illuminated by a light 1004 on the mobile device, and the mobile device held steady as a test is conducted. In the depicted embodiment, the mobile device 100 may be positioned near the face of the examinee, with the rear of the device 100 facing the examinee TT to allow a rear-facing camera 1002 of the device to capture images of the examinee's eye(s).

As depicted in FIG. 1B, in order to take the examination, the mobile device needs to be maintained in the appropriate position near the face of the examinee TT. As depicted, for some self-administered test embodiments, the examinee TT may hold the device 100 steady by bracing their elbow on a surface such as a table T while holding the device 100 in the appropriate position. In other embodiments, a brace such as a selfie stick or a phone stand could be used. For proctored embodiments, the proctor could hold the device 100 or could use a selfie stick or suitable stand to retain the device 100 in position.

With many mobile devices, the rear-facing camera has higher resolution than the front-facing camera, where present, allowing higher quality images to be collected. In performing a test using the rear-facing camera 1002, having the examinee TT face the back side of the device 100 may also reduce distractions. The mobile device may be positioned with the rear-facing camera towards the lower end and the examinee's gaze to be directed (as depicted at 1020) to a point 1006 above the camera, allowing the light 1006 to illuminate the face so that higher quality images may be captured without causing discomfort to the examinee. In some embodiments, the light 1006 may be operated at a reduced illumination, such as in range of from about 5% to about 50%, in range of from about 10% to about 35%, or in a range of about 10% or in a range of about 25% to obtain sufficient illumination while reducing potential discomfort. It will be appreciated that the particular light 1006 on the particular device 100 may determine the particular percentage required. For devices in which the light 1006 cannot be operated at a suitable reduced illumination, the examinee or proctor may be directed to place a semitransparent film or tape over the light 1006 to effectuate a similar appropriate level of light.

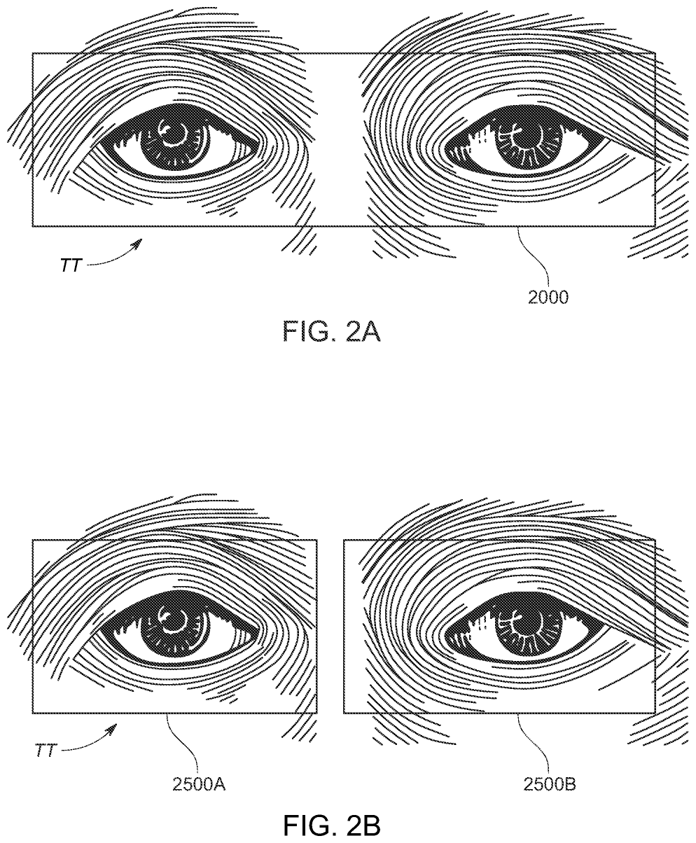

As depicted in FIGS. 2A and 2B, the mobile device may use facial recognition to determine when the eye or eyes of the user are within view of the camera 1002 and capture images of the eyes as an examination is conducted. In some embodiments, this may be accomplished by capturing images of a region of the face containing one or both eyes. As depicted in box 2000 of FIG. 2A, in some embodiments such a region may include both eyes, while in others it may include a single eye, as depicted at box 2500A or 2500B of FIG. 2B. The system may detect when the camera 1002 is in position to capture suitable images before allowing the test to commence and may monitor the position during an examination, pausing the test and directing the device 100 to be repositioned if the eyes of the examinee TT are no longer in proper position with respect to camera 1002. For proctored embodiments, the screen of the mobile device could display an image similar to FIG. 2A or FIG. 2B for viewing by the proctor while the rear of the device 100 faces examinee TT. A graphic representation of the required region for monitoring, similar to the depicted boxes 2000, 2500A or 2500B may be displayed, allowing the proctor to adjust the position of the device 100 as needed.

For use, a set of pre-test instructions may be provided to the examinee TT. Such instructions could include instructions for proper positioning of mobile device 100, including confirmation when proper positioning is detected. It may also include one or more short practice sessions to familiarize the examinee TT with the testing process. The instructions may be provided as audio instructions using a speaker of mobile device 100.

For testing, a test protocol for deception detection may be used to audibly present statements to the examinee TT using the audio output device, which may be an automated protocol utilizing statements taken from a database. For example, in some embodiments an audio multi-issue comparison test (AMCT) protocol may be used. Examples of some suitable AMCT test protocols may be similar to those disclosed in U.S. patent application Ser. No. 17/336,639 filed Jun. 2, 2021 and published as US Patent Application Publication US 2021/0369162, the contents of which are incorporated herein in its entirety.

In AMCT-type examinations conducted in accordance with the principles of the present disclosure, the examinee TT may be instructed to answer quickly and accurately to avoid failing the test. Questions may be asked by presenting a series of statements, which each require a “True” or “False” response. The statements may be about a number of different issues that are explained to the examinee TT as being relevant to the examinee, with at least one issue being a target behavior or issue of interest.

In some embodiments, the protocol may test up to four relevant issues, of which one, two, or three relevant issues may be of primary concern, and the other relevant issue(s) used for comparison. For example, each of relevant issues could address the examinee's participation in target behaviors or issues of concern for an employment position. In an illustrative example of a relationship fidelity test, the primary issue of concern could be infidelity, with the remaining issues being relevant issues with a lower prior probability of guilt. It will be appreciated that these examples used herein are merely illustrative, and any type of target behavior may be examined. When considering the target behaviors to address with the test, it is important to be as specific as possible to eliminate any uncertainty for the examinee TT. The issues that are not of primary concern should meet the following criteria: 1) be a crime more serious than the other relevant issues. 2) not cross over with the other relevant issues (should not be a related topic), 3) have face validity for the examinee (i.e., the examinee must believe the issue is important), and 4) have a lower expected prior probability of guilt. In some preferred embodiments, only a single relevant issue may be of primary concern with the other issues serving for comparison.

The statements may be presented by the device 100 aurally using the speaker during the examination. Each statement requires the examinee TT to verbally provide a “True” or “False” response which is captured by the microphone of the mobile device 100. In some embodiments, the statements are structured such that the examinee does not know to answer True or False until the end of the question. The statements may be presented such that no statements about the same issue are presented sequentially, and the statements may be balanced for length and negation. After receiving a response from an examinee, the mobile device 100 may produce an audio queue to indicate an answer has been received before moving on to the next question.

The statements may additionally vary in phrasing, asking a subsequent question in in a different way than the previous question. Sometimes, a question may repeat the subject matter of a prior question but phrased in an inverted manner. In a non-limiting example, in one test, an examinee may receive the question “on the topic of stealing the cell phone, I did not do it.” Several questions later, in the same test, the examinee may be asked “on the topic of the cell phone, I stole it.” An examinee may be expected to answer “true” to the former question and “false” to the latter question.

In other embodiments, a Directed Lie Comparison (DLC) test protocol may be used. In DLC-type examinations conducted in accordance with the principles of the present disclosure, the examinee TT may be instructed to answer quickly and accurately to avoid failing the test. Questions may be asked by presenting a series of statements, each requiring a “True” or “False” response. The statements may be about a number of different issues that are explained to the examinee TT as being relevant to the examine, with one issue being a target behavior or issue of interest, others may be Directed Lie statements, and others regarding neutral topics, such as simple arithmetic. Directed Lie statements relate to minor transgressions to which all are guilty. Some examples include:

In my entire life, I have not made a mistake.

In my entire life, I have always kept the rules.

In my entire life, I have never lied to someone.

The examinee is directed to lie in response to the Directed Lie statements, and the instructions inform the examinee that directed lie statements reveal how the eyes react when lying, and if the examinee does not react to those directed lie statements, they will fail the test.

In general, the DLC test predicts how examinees might react to relevant and directed lie statements. It predicts that innocent people will react more to the directed lie statements to avoid failing the test. Conversely, it predicts that guilty people will react more to the relevant statements because they pertain directly to the matter under investigation.

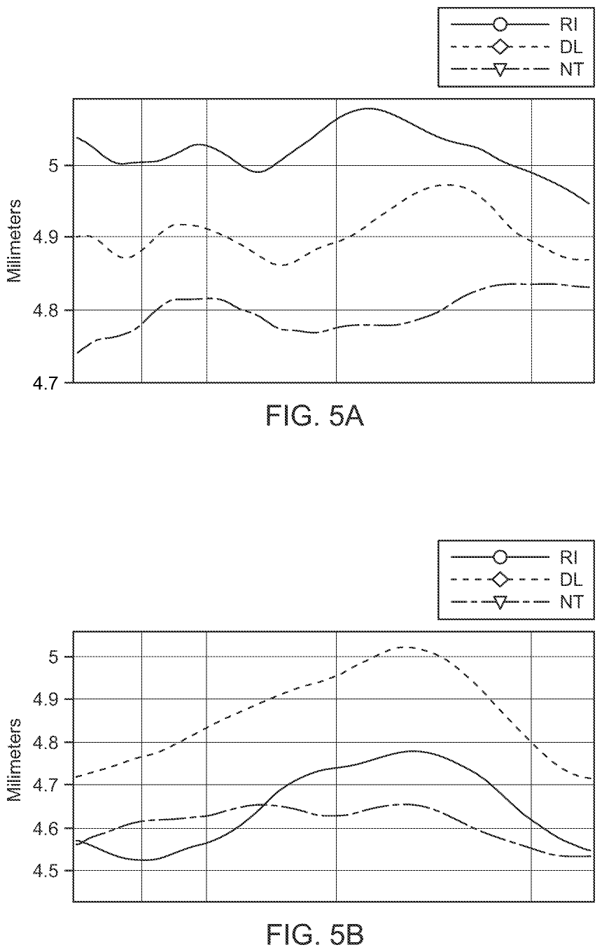

Response curves of pupil dilations in Not Credible vs. Credible examinee groups are shown FIGS. 5A and 5B. The change in pupil dilation (y-axis) is plotted against time (x-axis) to show pupil changes during responses to statement regarding Relevant Issue R1, the directed lie DL and a neutral topic NT. As shown, subjects determined to be Not Credible (FIG. 5A) show greater reaction with respect to the relevant issue, while Credible Subjects show the greatest reaction with respect to statements about the directed lie topics (FIG. 5B).

During the test, the mobile device captures measurements of features that may be used to discriminate truthful and nontruthful behavior. For example, the camera 1002 may be used to capture a series of images allowing the measurement and recordation of eye behaviors such as pupil dilation, eye movement, and vascular activity as the True and False statements are presented and statement responses recorded. Similarly, the response times may also be recorded.

In some embodiments, the camera 1002 may be used to take time-stamped images of the eyes of the examinee TT. For example, the mobile device 100 may be programmed to record images of the eyes at a desired rate, such as a rate of 30 frames per second. This allows the dilation of the pupils, movement of the eyes, and vasoconstriction to be determined by measuring changes in successive images over time.

Vascular activity may be determined by applying an image filter to the collected images so that only the channel of the color red is present in an image. The differences between the time-stamped filtered images may then be compared to allow a relative measurement of vascular activity, such as blood flow and/or blood pressure, to be correlated with the presentation of statements and the examinee's responses.

The system may pause the test and alert the examinee if the examinee eyes are no longer in view of the camera. Such an alert may be an audible prompt or warning sound.

At the conclusion of the test, the recorded measurements may be analyzed by means of a decision model to compute the probability of credibility or deception. In some embodiments, this may be performed by the mobile device. In the typical embodiment, however, the data taken in each test are encrypted and then synchronized with a secure web server that is part of the system.

In some embodiments, the decision model may be used to generate a credibility score using a binary logistic regression equation, which is a statistical method for analyzing a data set with one or more independent variables. The equation may be represented as Pr (Truthful)=1/(1+exp (b₀+b₁X₁+b₂X₂+ . . . +bₖXₖ) where X is an ocular-motor characteristic or response time (variable) and b is an optimal weight for separating truthful and deceptive people. A Credibility score may be Pr(Truthful)×100. A decision rule may be: If Credibility score<50, then “deceptive”, otherwise, “truthful” The resulting calculation yields a binary outcome. In some cases, the test may be considered inconclusive if the Credibility score falls between two cutoffs, such as 45 to 55.

Some of the independent variables considered in the algorithm include pupil dilation, vascular activity, eye movement, and response time, among others. The aim of the equation is to obtain an accurate decision that depends on the relationship between the test subject's deceptive status and the set of independent variables considered-pupil dilation, eye movement, gaze, response time, fixations, etc. It will be appreciated that different values for determining the Credible/Not Credible boundary may be used for particular applications.

### EXPERIMENT

A mock crime experiment was conducted which was modeled after Cook et al. (Cook, A. E., Hacker, D. J., Webb, A. K., Osher, D., Kristjansson, S., Woltz, D. J., & Kircher, J. C. (2012). Lyin′ Eyes: Ocular-motor Measures of Reading Reveal Deception. Journal of Experimental Psychology: Applied, 18(3), 301-313), the contents of which are incorporated herein by reference. The experiment was conducted to collect the ocular-motor data needed to develop and cross-validate a statistical model of ocular-motor measures that computes a credibility score for a single issue. In this experiment, there were three relevant issues: (1) theft of $20, (2) theft of a ring, and (3) theft of a cell phone.

One hundred and thirty (130) subjects were recruited from the local community. They were told some subjects would commit one or more of the thefts, whereas others would be innocent and would not commit any of the crimes. Subjects were arbitrarily assigned to one of two groups. Guilty subjects stole $20 (n=67). The other group of subjects was innocent of all three crimes (n=63).

After subjects completed their instructions, they were given a credibility test, as examinees, in accordance with the present disclosure using either Android or iPhone devices operating an app to present the examination and collect data as discussed elsewhere herein. Instructions and a brief practice session were conducted. The examination contained 24 True/False statements about each of the three relevant issues (72 statements total). A TTS voice presented instructions and test statements aurally using the phone speaker while the camera was used to collect ocular data by collecting images as discussed previously herein. The mobile device also recorded response times and the number of questions answered incorrectly. No two questions about the same issue were presented sequentially, the questions were balanced for length and negation, and the participants did not know whether to answer True or False until the end of each statement. The expected (exculpating) answer was True for half the statements and False for the remaining statements. Subjects were told they should respond quickly and accurately to the statements or they would fail the test.

### Analysis

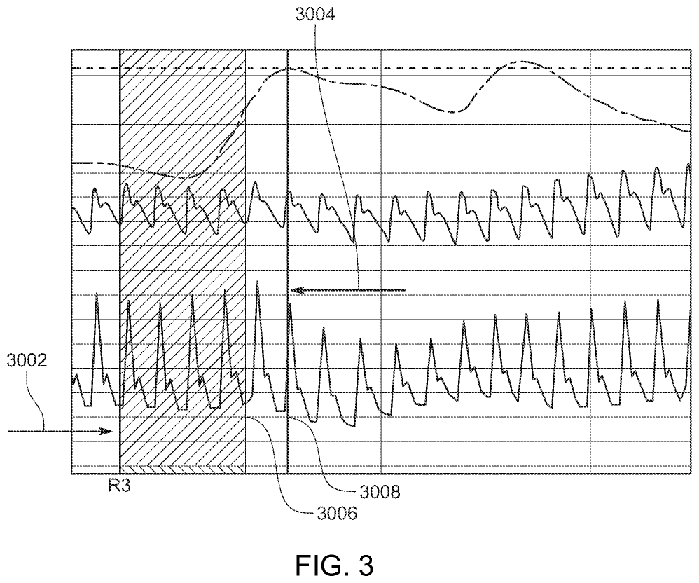

FIG. 3 depicts the measurement of reactions. Each reaction can be measured as depicted between arrows 3002 and 3004 as being relative to (1) Question onset (generally indicated at 3002), (2) Question offset (at 3006), and (3) and Subject answer (at 3008.) Question onset and subject's answer are used for tracking ocular measurements and question onset or offset is used for response time (RT).

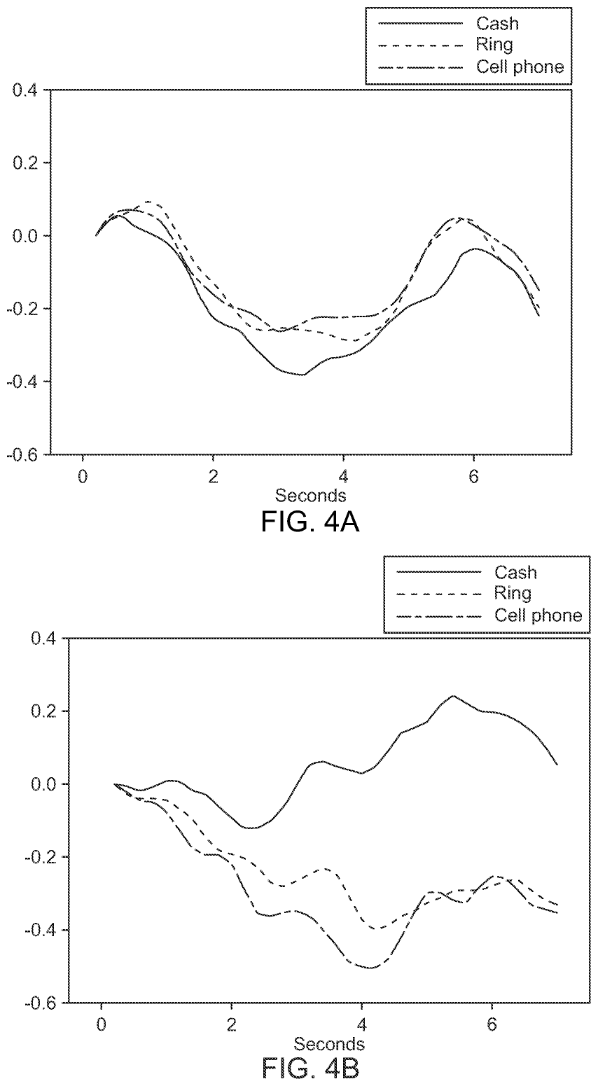

Mean pupil reactions in the innocent and guilty groups are shown FIGS. 4A and 4B relative to question onset. The change in pupil size (y-axis) is plotted against time (x-axis) to show pupil changes from question onset for seven seconds. As expected, innocent subjects (FIG. 4A) show little difference in pupil reactions to questions about the three relevant issues, whereas guilty subjects (FIG. 4B) react more to questions about stealing the cash.

Table 1 sets forth Validity Coefficients (point-biserial correlations) for features extracted from the data collected during the test. These extracted features were analyzed to measure how well they discriminate between truthful and deceptive people. The closer the score to +1 or −1, the more diagnostic it is, that is the better it discriminates between truthful and deceptive people. A validity coefficient of 0 is not diagnostic. As shown, all of these features were statistically significant, although pupil features were more diagnostic than vasomotor, eye movement, or response time.

TABLE 1

| Feature | Validity Coefficient |
|---|---|
| Pupil Dilation 01 | .52 |
| Pupil Dilation 02 | .35 |
| Vascular Activity | −.21 |
| Eye Movement | .20 |
| Response Time | −.24 |

These features were used to develop a decision model, as the following binary logistic regression model:

$$Pr ( Truthful ) = 1 / ( 1 + exp ( {b}_{0} + {b}_{1} {X}_{1} + {b}_{2} {X}_{2} + … + {b}_{k} {X}_{k} ) )$$

where X's comprise a subset of ocular-motor measures, and where b's are optimal weights for separating truthful & deceptive people.

Credibility score=Pr(Truthful)×100

If credibility<50, then “deceptive.” Otherwise, “truthful.”

Using this model, a 4-fold validation was performed. The sample of 130 subjects was split into 4 folds (subsets) with approximately equal numbers of guilty and innocent subjects. The validation was performed as follows. The first subset of 33 cases was omitted as a holdout sample and a model developed on the remaining 3 subsamples. Credibility scores were then computed credibility for subjects in the holdout sample and then accuracy recorded. The process was then repeated for the second subsample, with the model developed on subsamples 1, 3, and 4, then the third subsample and then fourth being used as the holdout.

Table 2 shows percent correct for each fold, as well as the mean accuracy across the four folds.

TABLE 2

| | Fold | Fold | Fold | Fold | | |
|---|---|---|---|---|---|---|
| | 1 | 2 | 3 | 4 | Mean | Mean |
| n | 33 | 33 | 33 | 31 | | 130 (total N) |
| Innocent | 81.3 | 75.0 | 81.3 | 80.0 | 79.4 | |
| Guilty | 76.5 | 82.4 | 88.2 | 68.8 | 79.0 | |
| | | | | | Mean Accuracy | 79.2 |

While this disclosure has been described using certain embodiments, it can be further modified while keeping within its spirit and scope. This application is therefore intended to cover any variations, uses, or adaptations of the disclosure using its general principles. This application is intended to cover any and all such departures from the present disclosure as come within known or customary practices in the art to which it pertains, and which fall within the limits of the appended claims.

What is claimed is:

1. A system for credibility analysis including:
a mobile device comprising
at least a first processor, the at least first processor comprising software instructions that, when executed, are configured to present to a subject one or more statements or questions, as part of a credibility test, prompting a response from the subject;
an eye tracking device connected to the at least first processor comprising:
a camera that is configured to obtain oculomotor activity of the subject; and
a light source;
an audio input device connected to the at least first processor positioned to receive a verbal response from the subject;
wherein the at least first processor derives oculomotor data from the oculomotor activity obtained by the camera;
a credibility processor comprising software instruction to analyze the assessment protocol and compute a probability of deception from the oculomotor data and the prompted response.

2. The system for credibility analysis of claim 1, wherein the audio output device is configured to produce a sound after receiving the verbal response from the subject.

3. The system for credibility analysis of claim 1, wherein in the one or more oculomotor activities tracked by the eye-tracking device comprises tracking one or more of pupil dilation, eye movement, or vascular activity of the subject.

4. The system for credibility analysis of claim 1, wherein a response time of the verbal response by the subject is tracked by the at least first processor.

5. The system for credibility analysis of claim 1, the one or more statements or questions presented by the processor includes one or more of an introductory phrase, a topic phrase, or a declaration phase.

6. The system for credibility analysis of claim 1, wherein the light source is configured to illuminate within a range of about 10% to about 30% of a maximum illumination capability.

7. The system for credibility analysis of claim 1, wherein the camera of the eye tracking device is configured to track one or more oculomotor activities by tracking eye features during the credibility test by taking a series of pictures of at least one of the subject's eye during the credibility test.

8. The system for credibility analysis of claim 1, wherein the one or more statements or questions presented by the processor are focused on target behavior or issues.

9. The system for credibility analysis of claim 1, wherein the statements or questions repeat with inverted phrasing.

10. The system for credibility analysis of claim 1, wherein the audio output device produces an audible prompt when an eye of the subject is out of view of the eye tracking device.

11. The system for credibility analysis of claim 1, wherein the credibility processor is a processor to which the at least first processor transmits the collected images for feature extraction and credibility calculation.

12. A process for credibility analysis comprising:
transmitting, by one or more processors, one or more statements to a subject aurally using a mobile device, as part of a credibility test, prompting a response from the subject;
illuminating, by a light source, at least one eye of the subject with a light on the mobile device;
receiving with the mobile device, by the one or more processors, one or more responses to the one or more statements spoken by the subject;
receiving, by the one or more processors, one or more images of the at least one eye of the subject from a camera of the mobile device, wherein the one or more images comprise oculomotor activity;
generating, by the one or more processors, oculomotor data from the one or more images of the at least one eye of the subject based on the oculomotor activity;
determining, by the one or more processors, one or more oculomotor activities of the subject during the credibility test;
computing, by the one or more processor, an overall probability of deception from the determined oculomotor activities and the prompted response.

13. The process for credibility analysis of claim 12, further comprising producing, by an audio output device, a sound after receiving the one or more responses from the subject.

14. The process for credibility analysis of claim 12, wherein in the one or more oculomotor activities tracked by the eye-tracking device comprises tracking one or more of pupil dilation, eye movement, or vascular activity of the subject.

15. The process for credibility analysis of claim 12, wherein a response time of the verbal response by the subject is tracked by the processor.

16. The system for credibility analysis of claim 12, the one or more statements or questions presented by the processor includes one or more of an introductory phrase, a topic phrase, or a declaration phase.

17. The process for credibility analysis of claim 12, wherein the light source is configured to illuminate within a range of about 15% to about 50% of a maximum illumination capability.

18. The process for credibility analysis of claim 12, wherein receiving, by the one or more processors, one or more images of at least one eye of the subject from a camera of the mobile device comprises taking a series of pictures of at least one eye during examination.

19. The process for credibility analysis of claim 12, wherein the one or more statements or questions presented by the process are focused on target behavior or issues.

20. The process for credibility analysis of claim 12, wherein the statements or questions repeat with inverted phrasing.

21. The process for credibility analysis of claim 12, wherein the audio output device produces a sound when the at least one eye of the subject is out of view of the eye tracking device.
